(19) United States
(12) Patent Application Publication
(10) Pub. No.: US 2008/0311196 A1
(43) Pub. Date: Dec. 18, 2008

(54) All Day Rhinitic Condition Treatment Regimen

(76) Inventor: Donna F. White, Louisville, KY (US)

Correspondence Address: INTELLECTUAL PROPERTY / TECHNOLOGY LAW, PO BOX 14329, RESEARCH TRIANGLE PARK, NC, 27709, US

(21) Appl. No.: 12/138,381

(22) Filed: Jun. 12, 2008

Related U.S. Application Data
(63) Continuation-in-part of application No. 11/485,931, filed on Jul. 13, 2006.

(51) Int. Cl. A61K 9/22; A61K 31/44; A61K 31/135; A61K 31/435; A61K 31/439; A61P 11/02

(52) U.S. Cl. 424/465; 514/357; 514/648; 514/291

(57) ABSTRACT

A therapeutic regimen is disclosed. The regimen includes a first pharmaceutical dosage form comprising a first therapeutically effective amount of a first antihistamine and an anticholinergic agent; and a second pharmaceutical dosage form comprising a therapeutically effective amount of a second antihistamine and an anticholinergic agent. The second antihistamine either has greater H₁ receptor activity or greater sedative effect than the first antihistamine, or is a different dose of the same antihistamine. The regimen comprises indicia for distinguishing between the first and second pharmaceutical dosage forms, wherein one dosage form is for daytime administration and the other is for nighttime administration. The regimen is placed in a prepackaged dispenser prefilled with the pharmaceutical dosage forms and comprising the indicia.

### CROSS-REFERENCE TO RELATED APPLICATIONS

This application is a continuation-in-part of U.S. patent application Ser. No. 11/485,931 for “ALL DAY RHINITIC CONDITION TREATMENT REGIMEN” filed Jul. 13, 2006.

### FIELD OF THE INVENTION

The present invention relates to rhinitis treatment regimens, more particularly, to prepackaged therapeutic regimens for the symptomatic treatment of rhinitic conditions which combine antihistamine and anticholinergic active agents in a manner so as to avoid undesired augmentation of hypertension in a subject.

### BACKGROUND OF THE INVENTION

Two types of oral medication are commonly used to treat rhinitis: decongestants and antihistamines. Decongestants and antihistamines differ in mechanism of action, therapeutic effects, and side effects. Many commercial rhinitis remedies combine the use of decongestants and antihistamines to bring about more complete symptom relief of rhinitis than with either medicament alone. Failure to treat symptoms of rhinitis may lead to other disorders including infection of the sinuses, ears and lower respiratory tract.

Decongestants are available both orally and topically as either short or long-acting preparations. Often combined with antihistamines, decongestants may help offset the soporitic effect of antihistamines. Decongestants may be tolerated during the day, and even be considered desirable to counter soporific effects, which are known to accompany other symptoms of rhinitis. Decongestants, however, may produce nervousness, restlessness, and insomnia if taken at night. This may confuse individuals to mistakenly attribute their inability to sleep to the malaise accompanying rhinitis rather than to the decongestant medication. Moreover, decongestants, for example, phenylephrine, pseudoephedrine and phenylpropanolamine, produce a narrowing of blood vessels which on one hand may lead to clearing of nasal congestion, but on the other hand, may cause an increase in blood pressure in patients who have high blood pressure.

Decongestants commonly used to treat rhinitis are predominately sympathomimetic agents. Sympathomimetic agents are generally a class of drugs whose pharmacology may include mimicking a stimulated sympathetic central nervous system. The effect of sympathomimetic agents may include increased cardiac output, dilation of bronchioles, and constriction of blood vessels. Sympathomimetic decongestants include, for example phenylephrine, pseudoephedrine and phenylpropanolamine. These agents act to constrict vessels in the nasal mucus membranes and thereby decrease tissue swelling and nasal congestion. In recent years certain sympathomimetic decongestants, for example, phenylpropanolamine, have been removed from OTC formulations because of implicated causation with hemorrhagic stroke.

Decongestants may be better than antihistamines for restoring the patency of congested nasal airways. However, like adrenaline, sympathomimetic nasal decongestants are stimulatory and generally may produce undesirable side effects. These undesirable side effects may include high blood pressure, heart irregularity and a general inability to tolerate decongestants.

Histamine is a mediator released from cells which line the walls of the nasal mucous membranes (mast cells). When released, histamine is known to bind to local receptors and thereby cause sneezing, nasal itching, swelling of the nasal membranes, and increased nasal secretions. Antihistamines relieve these effects, albeit by a different mechanism than decongestants. Antihistamines block the binding of histamine to histamine receptors in the nasal membranes, and thus function as H₁ receptor antagonists or inverse agonists. Antihistamines preemptively bind to histamine receptors and are effective only if given prior to histamine release because once histamine is released and binds to the receptor, administration of an antihistamine is generally ineffective. Although individuals may typically take antihistamines after symptoms occur, it is more desirable to dose antihistamine so as to effect therapeutic activity in anticipation of the peak times of histamine release. Individuals with rhinitic conditions commonly experience peak symptoms in the morning hours on awakening, a time believed to be concomitant with peak histamine release and coinciding with peak exposure to airborne allergens which would generally stimulate histamine release in sensitive individuals.

Anticholinergics generally block impulses from the part of the nervous system that controls heartbeat, blood pressure, and other responses to stress, by neutralizing the effects of acetylcholine. Anticholinergics include, for example, atropine, hyoscyamine, glycopyrrolate, methscopolamine, and scopolamine. Anticholinergics may help produce a drying effect in the nose and chest. Methscopolamine nitrate is a quaternary ammonium derivative of scopolamine, which possesses the peripheral actions of the belladonna alkaloids, but lacks the ability to cross the blood brain barrier, which is desirable. Methscopolamine nitrate may be used in combination with antihistamine because of its antisecretory effects on the respiratory system.

The combining of decongestants and antihistamines are generally known. The combination of decongestants and antihistamines may utilize a mechanistic approach where, for example, the incorporation of sympathomimetic decongestant and sedating antihistamine into a single dosage attempts to balance sympathomimetic stimulation and sedation of the respective components to possibly provide more complete relief of rhinitic symptomology than therapy with either component alone. Consequently, many products have been formulated so that their pharmaceutical dosage forms contain both a sympathomimetic decongestant and a sedating antihistamine.

While individuals are known to vary in their susceptibility to side effects, the incorporation of sympathomimetic decongestants and sedating antihistamine into a single dosage may cause some individuals to experience increased hypertension, irritability and/or sedation with these combinations. Examples of commercial formulations containing sympathomimetic decongestant and a sedating antihistamine include for example, BROMFED®, BENADRYL® and TAVIST-D®.

Formulations which incorporate both a sympathomimetic decongestant and a non-sedating antihistamine into a single dosage are generally known. While such formulations offer the advantage in being non-sedating, such combinations might be expected to provoke a greater incidence of risk to subjects with hypertension and may also exacerbate nighttime irritability and insomnia because the stimulating side effects of the decongestant may not be attenuated by concomitant use of sedating antihistamine. By way of example, the non-sedating antihistamine terfenadine, an active ingredient of SELDANE®, was removed from the market. A 25% incidence of insomnia has been reported among users of the combination of the non-sedating antihistamine terfenadine and the sympathomimetic pseudoephedrine decongestant. Examples of formulations including sympathomimetic decongestants and non-sedating antihistamines include, for example, CLARITIN-D®, CLARINEX-D® and ALLEGRA-D™.

Prepackaged regimens for treating the symptoms of rhinitis have been disclosed which employ a sympathomimetic decongestant for daytime and a decongestant plus a sedating antihistamine for night. See for example, U.S. Pat. No. 4,295,567, issued to Knudsen. Commercially available examples of the just mentioned regimens include: ACTIFED® and CONTAC DAY & NIGHT ALLERGY/SINUS®, both of which contains a sympathomimetic decongestant and a sedative antihistamine.

Prepackaged regimens have also been disclosed which incorporate a decongestant for daytime but not for nighttime, for example, SYN-RX™, which contains a sympathomimetic decongestant in the day formulation, and a non-sympathomimetic decongestant in the nighttime formulation. These regimens avoid stimulation from decongestant at night, however, they lack an antihistamine. Further, they neither contain medication which would be effective for rhinitis symptoms at night, nor anticipate peak rhinitic symptoms in the morning hours on awakening or formulate antihistamine pharmacological profiles to maximize therapeutic effectiveness while minimizing sedative side effects.

The short duration of sedation in relation to the longer duration of symptom suppression would suggest dosing of a sedative antihistamine at bedtime as a way to anticipate peak morning histamine release, and effectively confer combined antihistamine and decongestant activity during the day without sedation and without dosing of sedating antihistamine during the day. The limiting of stimulating decongestants during the day may avoid the potential for stimulation and insomnia at night. Such dosage regimens are disclosed in U.S. Pat. No. 6,270,796, U.S. Pat. No. 6,651,816 and U.S. Pat. No. 6,843,372 to Weinstein. Weinstein, however, requires in the disclosed regimen a sympathomimetic decongestant in at least one of the dosages and thus fails to address the risk associated with administering these decongestants to individuals with hypertension.

Thus, while the problems of sedation and nighttime irritability and insomnia, associated with treatments combining decongestants and sedating antihistamines, have been addressed, the problems associated with avoiding hypertension while effecting rhintic relief have not. Indeed, all sympathomimetic decongestant and antihistamine regimens have the potential to cause hypertension in addition to irritability and insomnia at night.

Similarly, the problem of sedation with treatments combining sympathomimetic decongestants and sedating antihistamines might be addressed in single entity combinations which employ sympathomimetic decongestants and non-sedating antihistamines, the problem of hypertension would not. Formulations which incorporate sympathomimetic decongestant and non-sedating antihistamine into a single pharmaceutical dosage form may be more likely to produce hypertension in addition to irritability and insomnia at night than formulations without sympathomimetic decongestants.

It is well known that individuals with rhinitis utilize antihistamines and decongestants hundreds of millions of times a year. It is not uncommon for inappropriate choices to result in symptomatic worsening rather than improvement. Individuals often use sedating medication unknowingly or inappropriately. Sympathomimetic decongestants taken at night not only may produce insomnia in a sizable portion of users, but may also cause daytime irritability, fatigue, and malaise. Users may mistakenly ascribe such symptoms to rhinitis rather than to medication. Professional as well as consumer confusion is widely encountered with these medications and unnecessarily negative consequences occur both by self-selection and prescription. There is a present need for preformulated regimens which advantageously use antihistamines and anticholinergics without sympathomimetic decongestants for rhinitic conditions in a manner to circumvent this confusion, avoid hypertension as well as reduce undesirable daytime sedation and/or eliminate nighttime stimulation.

Adherence to medication therapy and prevention of medication error are considerable medical problems and may be improved by establishing simplicity, reducing confusion, and increasing convenience. The proposed use of a multiplicity of pharmaceutical dosage forms as a regimen may be associated with pharmaceutical dosage forms being confused with each other, inadvertently switched, lost, misplaced, or ignored. Another problem associated with treatment using a plurality of pharmaceutical dosage forms is the lack of indicia which distinguish, signify and verify the pharmaceutical dosage forms from each other and their use together.

With more and more Americans becoming hypertensive, the need for new or improved rhinitis treatment regimens is becoming more of an issue. As many people cannot take decongestants because of concomitant disease states such as high blood pressure, heart irregularity and inability to tolerate decongestants, the present invention addresses this concern via a combination of active agents and treatments for rhinitic conditions with reduced or eliminated adverse hypertensive side effects.

### SUMMARY OF THE INVENTION

Therapeutic regimens for treating rhinitis are provided. The regimens include daytime and nighttime formulations, each of which comprises an anticholinergic and an antihistamine. The daytime formulation and nighttime formulation either include different antihistamines, or include different dosages of the same antihistamine.

Where different dosages of the same antihistamine are used, the nighttime formulation can include a relatively higher dosage of an antihistamine than used in the daytime formulation. For example, a sedating antihistamine can be used in the nighttime formulation and a lower dose of the antihistamine can be used during the day. In this manner, one can largely avoid sedation during the day. To the extent the dosage of the antihistamine is lower during the day, a decongestant can be added to the daytime formulation, so long as there is a balance between the sedative properties of the antihistamine and the stimulatory properties of the decongestant, ideally such that the formulation is neither overly stimulatory nor overly sedative.

Where different antihistamines are used in the daytime and nighttime formulations, in one aspect of this embodiment, a second generation antihistamine is used in the daytime formulation, and a first generation antihistamine is used in the nighttime formulation. In this manner, one can avoid sedation during the day, but provide sedation at night. There may also be a different dosage between the first and second generation antihistamines, depending on the effective dosage for the selected antihistamines. In another aspect of this embodiment, the first antihistamine is less active than the second antihistamine. The term “less active” is used herein to describe lower dosages of the same antihistamine, with respect to the first embodiment, or with respect to the second embodiment, an antihistamine with less antagonistic activity at the histamine H1 receptor.

In one aspect of this embodiment, the second generation antihistamine is not acrivastine, astemizole, cetirizine, loratadine, mizolastine or fexofenadine. Because the effective dosage of different antihistamines differs depending on the antihistamine, the amount of antihistamine in each of the daytime and nighttime formulations may be the same or different.

In both of these embodiments, the therapeutic regimens include indicia for distinguishing between the first and second pharmaceutical dosage forms, where the indicia distinguishes the first pharmaceutical dosage form for daytime administration and the second pharmaceutical dosage form for nighttime administration. A prepackaged dispenser pre-filled with the pharmaceutical dosage forms and including the indicia is also provided.

In a third embodiment, rather than using a combination of a daytime and a nighttime formulation, a composition is provided in a single dosage form, which provides for sustained release of an anticholinergic agent, release of a first antihistamine for an initial twelve hour period, and delayed release of a second antihistamine for a second twelve hour period. This can be accomplished, for example, using a drug delivery device that includes a core and a coating, wherein the first antihistamine is present in the coating, and the second antihistamine is present in the core, with the anticholinergic agent present in both the core and the coating. The terms “first antihistamine” and “second antihistamine,” in this context, refer to either the differing dosages of the same antihistamine, or to a first generation and a second generation antihistamine, as discussed above with respect to the daytime and nighttime formulations. Thus, in this embodiment, one can achieve the same benefits as provided by taking both the daytime and nighttime formulations, but need only to take a single dose. The single dose form may be formulated for administration at night, by including a sedating antihistamine in the coating for instantaneous release and a non-sedating antihistamine in the core for delayed release. The single dose form may also be formulated for administration during the day, with a non-sedating antihistamine in the coating for initial sustained release, and a sedating antihistamine in the core for delayed sustained release. Both coating and core contain an anticholinergic for continuous sustained release. This embodiment includes a prepackaged dispenser with indicia describing the time of day for administration, depending on which antihistamine is initially released.

These therapeutic regimens can be used to treat the symptoms of a rhinitic condition. The presence of the anticholinergic in both the daytime and nighttime aids in suppressing post-nasal drip throughout the day and night. The evening administration of the anticholinergic agent can help avoid the potential complications of having mucous, particularly mucous infected with bacteria and/or viruses, enter the bronchial tubes and result in bronchitis. The daytime administration of the anticholinergic agent can help minimize post-nasal drip. Further, with respect to those embodiments where the daytime formulations include a second generation antihistamine, one difference between first and second generation antihistamines is that the first generation antihistamines tend to have some anticholinergic properties, but second generation antihistamines tend not to have such properties. Thus, the presence of an anticholinergic can provide anticholinergic properties that are lacking in the second generation antihistamine used in the daytime formulation.

In addition to the therapeutic regimens, and methods of treatment using the regimens, methods for formulating rhinitic treatment regimens are also provided. In one embodiment, the formulation method comprises producing a first pharmaceutical dosage form that includes a therapeutically effective amount of a first antihistamine and a therapeutically-effective amount of an anticholinergic agent, and a second pharmaceutical dosage form comprising a therapeutically-effective amount of a second antihistamine, and a therapeutically effective amount of the anticholinergic. The formulations are intended for use in a manner in which the first pharmaceutical dosage form is intended to be administered during the day, and the second pharmaceutical dosage form is intended to be administered during the evening. So that the proper formulations are administered, the formulations are marked with indicia for daytime and nighttime administration.

As discussed above, the daytime and nighttime formulations either include different antihistamines, or include different dosages of the same antihistamine. Where different dosages of the same antihistamine are used, the nighttime formulation can include a relatively higher dosage of an antihistamine, for example, a sedating antihistamine, than the daytime formulation. To the extent the dosage of the antihistamine is lower during the day, a decongestant can be added to the daytime formulation. Where different antihistamines are used in the daytime and nighttime formulations, in one aspect of this embodiment, a second generation antihistamine is used in the daytime formulation, and a first generation antihistamine is used in the nighttime formulation. In another aspect of this embodiment, the first antihistamine is less active than the second antihistamine. The term “less active” is used herein to describe lower dosages of the same antihistamine, with respect to the first embodiment, or an antihistamine with less antagonistic activity at the histamine H1 receptor, in the second embodiment.

In one aspect of this embodiment, the second generation antihistamine is not acrivastine, astemizole, cetirizine, loratadine, mizolastine or fexofenadine. Because the effective dosage of different antihistamines differs depending on the antihistamine, the amount of antihistamine in each of the daytime and nighttime formulations may be the same or different.

The methods for formulating these regimens further include providing indicia for distinguishing between the first and second pharmaceutical dosage forms, where the indicia distinguishes the first pharmaceutical dosage form for daytime administration and the second pharmaceutical dosage form for nighttime administration. A prepackaged dispenser pre-filled with the pharmaceutical dosage forms and including the indicia is also provided.

Alternatively, one can formulate a single dosage form which provides for sustained release of an anticholinergic agent, release of a first antihistamine for an initial twelve hour period, and delayed release of a second antihistamine for a second twelve hour period. This can be accomplished, for example, by formulating a drug delivery device that includes a core and a coating, wherein the first antihistamine is present in the coating, and the second antihistamine is present in the core, with the anticholinergic agent present in both the core and the coating.

The present invention will be better understood with reference to the following detailed description.

### BRIEF DESCRIPTION OF THE DRAWINGS

For a fuller understanding of the nature and object of the present invention, reference is made to the accompanying drawings, wherein:

FIG. 1 is a perspective view of the front side of an exemplary embodiment of a pharmaceutical dosage form dispenser.

### DETAILED DESCRIPTION

The therapeutic regimens for treating rhinitis, and methods of treatment using the regimens, are described in more detail below. The present invention will be better understood with reference to the following definitions:

As used herein, the term “anticholinergic” refers to a moiety that reduces or inhibits effects of acetylcholine on the central nervous system or peripheral nervous system. For example, moieties having as their main therapeutic effect reversible inhibition of muscarinic acetylcholine receptors or nicotinic acetylcholine receptors are considered anticholinergics. Anticholinergics with limited capacity to cross the blood-brain barrier are preferred, for example, methscopolamine salts and glycopyrrolate.

As used herein, the term “antihistamine” refers to a moiety that inhibits reduces effects mediated by histamine. By way of example, moieties having negative modulation of histamine receptors as their main therapeutic effect (i.e., antagonists at the histamine H1 receptor) are antihistamines. For example, chlorpheniramine, which may also have anticholinergic activity, is considered an antihistamine. Antihistamine includes, for example, first generation antihistamines such as ethylenediamines, ethanolamines, alkylamines, piperazines and tricyclics. Antihistamines further include second generation antihistamines such as acrivastine, astemizole, cetirizine, loratadine, mizolastine, desloratidine and fexofenadine.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

As used herein, the terms “daytime” and “nightime” are intended to include literally day and night, in that such times vary in accordance with the schedule of the individual.

As used herein, the term “H₁ receptor activity” refers to a level of inhibition of binding to or interacting with a H₁ receptor. The term H₁ receptor activity includes H₁ receptor antagonistic activity and H₁ receptor inverse agonistic activity. Determining H₁ receptor activity is known, see, e.g., V. T. Tran et al, “Histamine H₁ receptors identified in mammalian brain membranes with [³H]mepyramine”, Proc. Natl. Acad. Sci. (1978) 75: 6290-94. The Tran et al. document is incorporated by reference herein in its entirety.

As used herein, “pharmacologically active agent” or “active agent” are used interchangeably and refer to a compound or composition of matter that when administered to a subject (human or animal) causes a desired pharmacologic and/or physiologic effect by local and/or systemic action.

As used herein, “pharmaceutical dosage form” refers to a dosage form of an active agent (e.g., sublingual, chewable, buccal and extended delivery tablets (EDT) forms, tablets, capsules, lozenges, gelcaps, powders and oral care strips/film) which is generally safe, non-toxic and not biologically or otherwise undesirable. A pharmaceutical dosage form includes that which is acceptable for veterinary and/or human pharmaceutical use, and which possesses the necessary and desirable characteristics of a dosage form acceptable for administration to a subject (e.g., a tablet of acceptable hardness, dissolution, stability, and a size and weight practical for oral administration).

As used herein, “pharmacological effect” encompasses pharmacokinetic and pharmacodynamic effects produced in a subject that achieve the intended purpose of a therapy or regimen. By way of example, a pharmacological effect means that rhinitis symptoms of the subject being treated are prevented, alleviated, or reduced. In one example, a pharmacological effect would include inhibition of binding or interacting with a H₁ receptor. In another example, a pharmacological effect would include one that results in the prevention or reduction of hypertension in a treated subject.

As used herein, the term “rhinitic condition” refers to the symptomology of rhinitis. Symptomology of rhinitis includes, for example, repetitive sneezing, rhinorrhea (runny nose), post-nasal drip, nasal congestion, pruritic (itchy) eyes, ears, nose or throat, and generalized fatigue. Rhinitic condition may also include wheezing, eye tearing, sore throat, impaired smell, sinus headaches and ear plugging.

As used herein, “sedative effect” refers to a soporific property of an antihistamine. Determining a soporific property of an antihistamines is known, see e.g., Witek, et al., “Characterization of daytime sleepiness and psychomotor performance following H₁ receptor antagonists,” Annals of Allergy, Asthma, & Immunology (1995) 74: 419-26, the disclosure of which is incorporated by reference in its entirety.

The term “sleep” as used herein refers to a prolonged period of rest during which the individual exhibits decreased activity. Typically sleep is a period of rest lasting for 3 or more hours, more typically about 6-8 hours for most adult humans. The term “sleep” as used herein relates to periods of resting wherein all the clinical stages of sleep are not achieved as well as periods of rest wherein all the clinical stages of sleep are achieved. The term “awaken” or “awakening” relates to the physical condition of arousal from sleeping or resting, and is characterized by an increase in the level of physical activity. The period of awakening is generally understood to occur from about 4 or more hours after the commencement of sleep.

As used herein, “sympathomimetic decongestant” refers generally to a class of decongestant whose pharmacology includes mimicking a stimulated sympathetic central nervous system. The effect of sympathomimetic decongestant includes, for example, increased cardiac output, dilation of bronchioles, and constriction of blood vessels. Sympathomimetic decongestants include, for example phenylephrine, pseudoephedrine and phenylpropanolamine.

### I. Treatment Regimens

The regimens include daytime and nighttime formulations, each of which comprises an anticholinergic and an antihistamine. The daytime formulation and nighttime formulation either include different antihistamines, or include different dosages of the same antihistamine.

Suitable antihistamines include both first and second generation antihistamines.

A. First Generation (Sedating) Antihistamines

First-generation H₁-antihistamines can be classified on the basis of chemical structure, and agents within these groups have similar properties. They all tend to be sedating, though some are more sedating than others. These compounds have antagonist properties at the histamine H₁-receptor, and also tend to also act as antagonists at muscarinic receptors, resulting in sedation and the drying of secretions in the airways.

The antimuscarinic effects of these compounds can be assessed, for example, on ion transport by mucus gland cells isolated from the airways of swine. Enzymatically isolated airway mucus gland cells are typically purified utilizing density gradients and grown in culture on porous inserts (Millicell HA™) at an air interface. Cells grown in this manner maintain phenotype and polarity. Transport of ions, as short-circuit current measured under voltage-clamp, can be measured in response to acetylcholine (ACh) or histamine applied to the serosal side of the gland cell layers. Concentration-response relationships for ACh or histamine can be generated in the presence and absence of various drugs. The potencies against muscarinic receptor activation can be estimated using the dose-ratio method of Schild.

Representative first generation antihistamines are listed below.

| Class | Description | Examples |
|---|---|---|
| Ethylenediamines | Ethylenediamines were the first group | Mepyramine |
| | of clinically-effective H₁- | (pyrilamine) |
| | antihistamines developed. | Antazoline |
| Ethanolamines | Diphenhydramine was the prototypical | Diphenhydramine |
| | agent in this group. Significant | Carbinoxamine |
| | anticholinergic adverse effects, as well | Doxylamine |
| | as sedation, are observed in this group | Clemastine |
| | but the incidence of gastrointestinal | Dimenhydrinate |
| | adverse effects is relatively low.^{[3][6]} | |
| Alkylamines | The isomerism is a significant factor in | Pheniramine |
| | the activity of the agents in this group. | Chlorphenamine |
| | E-triprolidine, for example, is 1000- | (chlorpheniramine) |
| | fold more potent than Z-triprolidine. | Dexchlorpheniramine |
| | This difference relates to the | Brompheniramine |
| | positioning and fit of the molecules in | Triprolidine |
| | the histamine H₁-receptor binding site. | |
| | ^{[6]}Alkylamines are considered to have | |
| | relatively fewer sedative and | |
| | gastrointestinal adverse effects, but | |
| | relatively greater incidence of | |
| | paradoxical CNS stimulation.^{[3]} | |
| Piperazines | These compounds are structurally- | Cyclizine |
| | related to the ethylenediamines and the | Chlorcyclizine |
| | ethanolamines, and produce significant | Hydroxyzine |
| | anticholinergic adverse effects. | Meclizine |
| | Compounds from this group are often | |
| | used for motion sickness, vertigo, | |
| | nausea, and vomiting. The second- | |
| | generation H₁-antihistamine cetirizine | |
| | also belongs to this chemical group. | |
| Tricyclics and | These compounds differ from the | Promethazine |
| Tetracyclics | phenothiazine antipsychotics in the | Alimemazine |
| | ring-substitution and chain | (trimeprazine) |
| | characteristics. (Nelson, 2002) They | Cyproheptadine |
| | are also structurally-related to the | Azatadine |
| | tricyclic antidepressants (and | Ketotifen |
| | tetracyclics), explaining the H₁- | |
| | antihistaminergic adverse effects of | |
| | those three drug classes and also the | |
| | poor tolerability profile of tricyclic H₁- | |
| | antihistamines. The second-generation | |
| | H₁-antihistamine loratadine was | |
| | derived from compounds in this group. | |

These compounds, and other first generation antihistamines, share common structural features. That is, they tend to include two aromatic rings, connected to a central carbon, nitrogen or CO moiety. They tend to include a spacer between the central carbon, nitrogen, or carbonyl moiety and the amine, usually 2-3 carbons in length, linear, ring, branched, saturated or unsaturated. The amine tends to be substituted with small alkyl groups, such as methyl. Where the central carbon moiety is chiral, the chirality can increase both the potency and selectivity for H1-receptors. For maximum potency, the two aromatic rings should be orientated in different planes. For example, tricyclic ring systems are slightly puckered, and the two aromatic rings lie in different geometrical planes, typically giving the corresponding drugs a very high potency.

This group of antihistamines, which have been in use for a long period of time, is well known to have the potential to cause sleepiness. Below is a list first generation antihistamines, which can have sedative properties.

Brompheniramine

Buclizine

Chlorpheniramine

Cinnarizine

Clemastine

Cyclizine

Cyproheptadine

Diphenhydramine

Diphenylpyraline

Doxylamine

Meclozine

Pheniramine

Promethazine

Triprolidine

B. Second Generation (Non-Sedating Antihistamines

Second generation antihistamines are known to provide less sedation, and are suitable for both the daytime and nighttime formulations. Of these second generation antihistamines, only desloratidine is also known to have decongestant properties. In some embodiments, it may be desirable to exclude desloratidine to avoid these properties.

Second generation H1-antihistamines are newer drugs that are much more selective for peripheral H1 receptors in preference to the central nervous system histaminergic and cholinergic receptors. This selectivity significantly reduces the occurrence of adverse drug reactions compared with first-generation agents, while still providing effective relief of allergic conditions.

Third-generation H1-antihistamines are the active enantiomer (levocetirizine) or metabolite (desloratadine & fexofenadine) derivatives of second-generation drugs, which are intended to have increased efficacy with fewer adverse drug reactions. Indeed, fexofenadine is associated with a decreased risk of cardiac arrhythmia compared to terfenadine.

A representative list of second-generation antihistamines is provided below:

Acrivastine

Astemizole

Cetirizine

Loratadine

Mizolastine

Terfenadine

Representative third-generation antihistamines include the following:

Levocetirizine

Desloratadine

Fexofenadine

C. Anticholinergics

Anticholinergic agents are exemplified by the belladonna alkaloids atropine and scopolamine, which inhibit the muscarinic action of acetylcholine on structures innervated by postganglionic cholinergic nerves. These agents typically inhibit the nasal secretory mechanism and dry the nasal membranes. Anticholinergic agents also are known to exert central effects such as papillary dilatation and CNS stimulation and depression.

Attention to the central effects of the anticholinergic agent considered and the relationship of the amount of dosage to central effects are of importance in devising a non-sedating oral dosage unit for rhinitis. Drowsiness is known to occur with high doses of anticholinergic agents, and with therapeutic doses of oral scopolamine, but drowsiness is considered rare with therapeutic doses of other oral anticholinergic agents (USPDI Drug Information for the Health Care Professional, 16th Edition, United States Pharmacopoeia Convention, Inc., 219-235, 1996 Rand McNally, Taunton, Mass.). Further, anticholinergic pharmaceuticals have been developed which have a limited capacity to pass across lipid membranes, such as the blood-brain barrier, and therefore have a limited capacity to produce central effects. Examples of these agents are the quaternary ammonium compounds methscopolamine and glycopyrrolate.

Scopolamine acts as a competitive antagonist at specific muscarinic acetylcholine receptors, specifically Ml receptors; it is thus classified as an anticholinergic, or, more specifically, as an anti-muscarinic drug.

The combination of an antihistamine and anticholinergic agent as described herein can dry the nasal passages via a different receptor than the nasal decongestant receptors used in the past.

D. Decongestants

A nasal decongestant is defined as a drug that shrinks the swollen membranes in the nose, making it easier to breath. The principle mechanism of action of a sympathomimetic decongestants is indirect action on the adrenergic receptor system. While it may have weak agonist activity at α- and β-adrenergic receptors, the principal mechanism is to displace norepinephrine from storage vesicles in presynaptic neurons. The displaced noradrenaline is released into the neuronal synapse where it is free to activate the postsynaptic adrenergic receptors. The response from adrenoreceptor al, chiefly responsible for vasoconstriction, decreases swelling in the membranes of the nose.

Representative decongestants that can optionally be present in the daytime formulations disclosed herein include sympathomimetic amines such as ephedrine, pseudoephedrine and phenylpropanolamine. The anticholinergics included in the dosage forms affect different receptors, namely the muscarinic receptors.

E. Optional Additional Components

In addition to antihistamines and anticholinergic active agents, other therapeutic ingredients for the treatment of rhinitis may be formulated if desired. For example, analgesics such as salicylates and acetaminophen may be included. In addition to the pharmacologically active agents, pharmaceutical dosage forms may contain a number of inert materials or additives. Inert materials and additives may include materials that help in the manufacture of the tablet or to impart satisfactory compression characteristics to the formulation. Inert materials and additives may also include materials that help to give additional desirable physical characteristics to the dosage form, such as colors, flavors, and sweetening agents. Such inert materials and additives should not materially affect the pharmacological properties of the active agent or agents.

Pharmaceutical dosage forms may contain one or more excipients or vehicles chosen from diluents, lubricants, binders, disintegrating agents, absorbents, and the like. The dissolution rate of a pharmaceutical dosage form may be increased by the addition of disintegrant or solubilizing substances.

### II. Formulations

Tablets

Individual tablets for the daytime and nighttime formulations can be prepared by any suitable tabletting technique known to those skilled in the art. For example, the pharmaceutically active ingredient can be admixed with the excipient(s) and, if present, the disintegration enhancing agent, and advantageously formed into a tablet using a conventional tabletting press, or dosed into a capsule.

Where controlled release is desired, one can use conventional techniques within each tablet to control the release of the active agent. For example, representative controlled release formulations are described, for example, in U.S. publication 20040091544.

Another element of the composition is the controlled-release matrix system within which the active agents are dispersed. The controlled-release matrix system refers to a system containing a matrix, which consists of a continuum of material, and a controlled-release component which is contained by the matrix and which is present in an amount sufficient to provide a highly predictable pre-selected release profile of the active agents. The controlled release component is preferably finely dispersed throughout the matrix. The active agents are preferably finely dispersed throughout the controlled-release matrix system.

Controlled-release can be achieved by including one or more ingredients for controlling the rate at which the active components are made available to the biological system of a host. The controlled-release components can include a sustained release ingredient or a combination of at least two of the following ingredients: an instantaneous release ingredient, a delayed release ingredient or a sustained release ingredient.

An instantaneous release ingredient is self-explanatory in that it refers to an ingredient which promotes or enhances immediate release of the active agents. The instantaneous release ingredient can be an additional ingredient which enhances dispersion of the active agents, for example, a surfactant.

A delayed release ingredient is an ingredient which prevents the active agents from being made available until some time after initial administration. Examples of delayed release ingredients include, but are not limited to, polymeric or biodegradable coatings or matrices, including water soluble cellulose polymers.

A sustained release ingredient is an ingredient, or combination of ingredients, which permits release of the active agents at a certain level over a period of time. Examples of sustained release ingredients include gels, waxes, fats, emulsifiers, combinations of fats and emulsifiers, polymers, starch, water soluble cellulose polymers, etc., as well as the above in combination with other polymeric or biodegradable coatings or matrices.

The controlled-release component may include at least one water soluble cellulose polymer, including a water soluble high molecular weight cellulose polymer. High molecular weight cellulose polymers are cellulose polymers with an average molecular weight of at between about 25,000 and about 85,000. The exact molecular weight cellulose polymer used will generally depend upon the desired release profile, and as a general rule, polymers having an average molecular weight of about 25,000 are useful in a controlled-release composition having a time release period of up to about 8 hours, while polymers having an average molecular weight of about 85,000 are useful in a controlled-release composition having a time released period of up to about 12 hours. Higher molecular weight cellulose polymers may provide release over a period longer than about 12 hours, and, therefore, would provide release of a daytime formulation in the nighttime, or a nighttime formulation in the daytime, so these are less preferred.

The controlled-release component preferably consists of a water soluble cellulose polymer, preferably a high molecular weight cellulose polymer, selected from the group consisting of hydroxypropyl methyl cellulose (HPMC), hydroxyethyl cellulose (HEC), hydroxypropyl cellulose (HPC), carboxy methyl cellulose (CMC), and mixtures thereof. Of these, the most preferred water soluble cellulose polymer is HPMC. Preferably the HPMC is a high molecular weight HPMC, with the specific molecular weight selected to provide the desired release profile, that is, typically between 30 minutes and 12 hours, more preferably, between one and eight hours.

The water soluble cellulose polymer, e.g., high molecular weight HPMC, is typically incorporated into the controlled-release matrix system as a fine particulate material having a particle size such that not less than 80% of the particles pass through an 80 mesh screen.

There are various ways to achieve a desired release profile. For example, the active agents can be associated physically (which also includes being chemically associated or bound) with the controlled-release component, within the controlled-release matrix system. Alternatively, the active agents can be coated, laminated, encapsulated, etc., with the controlled-release component, within the controlled-release matrix system. Regardless of the method of providing the desired release profile, the present invention contemplates using a controlled-release component containing one or more of the ingredients, as described above.

The controlled-release matrix system can be administered in the form of a liquid as a suspension or solution, or alternatively in solid form, such as a tablet, pellet, particle, capsule, or soft gel. For example, the form can be polymeric capsules filled with solid particles which can, in turn, be made to release the active agents according to a known pattern or profile. Such particles can also be made to have more than one release profile so that over an extended time the combined release patterns provide a pre-selected profile.

Preferably, the controlled-release matrix system combination is administered in the form of a heterogeneous matrix, such as, for example, a compressed tablet, to control the release of the active agents either by diffusion, erosion of the matrix or a combination of both.

Other combinations which are contemplated include a combination of polymeric material(s) and active agents which is formed into a sandwich, and which relies on diffusion or erosion to control release of the active agents. Additionally, heterogeneous dispersions or solutions of active agents in water-swellable hydrogel matrices are useful in controlling the release of the active agents by slow surface-to-center swelling of the matrix and subsequent release of the active agents by a combination of diffusion of the active agents from the water-swollen part of the matrix and erosion of the water-swollen matrix containing the active agents.

The controlled-release matrix system will preferably provide for a sustained release of active agents according to a desired release profile through the use of one or more of the release ingredients described above. More preferably, the controlled-release matrix system will provide a release profile which releases the active agents at a substantially constant rate over a designated time period.

As the terminology is used herein, “substantially constant rate” refers to maintaining a release rate of the active agents within a desired range over at least about 75% of the designated time period for release, preferably over at least about 80% and more preferably over at least about 90% of the designated time period. The desired range for release is preferably about 4.0+/−1.0 percent of the daily dosage of the active ingredient per hour, more preferably about 4.0+/−0.7 percent per hour and most preferably about 4.0+/−0.5 percent per hour. For example, a tablet for 12-hour timed-release of the active agents, which releases the active agents at a substantially constant rate, would maintain a release rate in the range of about relatively constant rate over at least 75 percent of the 12 hour period.

A release profile which provides for a substantially constant release rate of the active agents will result in a more consistent blood serum level of the active agents over the delivery period. As such, the amount of active agents delivered can be maximized, while avoiding the side effects attributable to high levels of active agents.

Such a release profile can typically be obtained by using a controlled-release matrix tablet, which contains hydroxypropyl methyl cellulose (HPMC) as the primary ingredient of the controlled-release component. The controlled-release component can also contain minor amounts of other materials which can affect the release profile. Examples of such materials include conventional waxes and waxy materials used in pharmaceutical formulations, such as carnuba wax, spermaceti wax, candelilla wax, cocoa butter, cetostearyl alcohol, beeswax, partially hydrogenated vegetable oils, ceresin, paraffin, myristyl alcohol, stearyl alcohol, cetyl alcohol and stearic acid. Hydrophilic gums are also contemplated for use, in minor amounts, which can have an effect on the release profile. Examples of hydrophilic gums include acacia, gelatin, tragacanth, veegum, xanthin gum, carboxymethyl cellulose (CMC), hydroxy propyl cellulose (HPC) and hydroxy ethyl cellulose (HEC).

Preferably, the HPMC in the controlled-release matrix tablet is a high molecular weight HPMC. The specific molecular weight used will typically vary depending upon the desired release profile. For example, a tablet designed to provide a substantially constant release rate over a 12 hour period will preferably contain HPMC having an average molecular weight of at least about 65,000, more preferably about 85,000.

Preferably, the controlled-release matrix tablet will contain about 4 to about 20 wt %, more preferably about 6 to about 16 wt % and most preferably about 8 to about 12 wt % HPMC. The exact amount of HPMC will vary depending upon the molecular weight of the HPMC and the desired release profile. For example, a tablet designed to provide a substantially constant release rate over a 12 hour period, which contains HPMC having a molecular weight of about 85,000, will preferably contain about 8 to about 12 wt %, more preferably about 10%, of the HPMC.

The HPMC used in making the controlled-release tablet will preferably be in the form of a fine particulate having a particle size such that not less than 80% of the HPMC passes through an 80 mesh screen.

The amount of active agents contained in the controlled-release tablet will preferably be an amount sufficient to provide a therapeutically effective dosage for an 8-12 hour period. Preferably, the controlled-release matrix tablet will provide a release profile which releases the active agents at a substantially constant rate over a designated time period.

Other ingredients can be used in accordance with the present invention to improve the tablet. The ingredients can be incorporated during the mixing stage, during the agglomeration stage or after the agglomeration stage. Such ingredients include binders, which contribute to the ease of formation and general quality of the tablet; lubricants, which aid in compressing and compacting the tablet; and flow agents or glidants, which adhere to the cohesive material in order to enhance flow properties by reducing interparticle friction.

Examples of useful binders include calcium sulfate, calcium carbonate, microcrystalline cellulose, starches, lactose, sucrose, mannitol, sorbitol, polyvinylpyrrolidone, methylcellulose, sodium carboxymethylcellulose, ethylcellulose, polyacrylamides, polyvinyloxoazolidone, and polyvinylalcohols. A preferred binder is microcrystalline cellulose, such as Avicel PH-101 sold by FMC Corporation.

Lubricants can include, but are not limited to, the following: magnesium stearate, calcium stearate, zinc stearate, stearic acid, hydrogenated vegetable oils, sterotex, polyoxyethylene, monostearate, talc, polyethyleneglycol, sodium benzoate, sodium lauryl sulfate, magnesium lauryl sulfate and light mineral oil. Of these, the preferred lubricants are magnesium stearate and stearic acid.

Flow agents or glidants which can be used include starch, talc, magnesium and calcium stearate, zinc stearate, dibasic calcium phosphate, magnesium carbonate, magnesium oxide, calcium silicate, silicon dioxide and silica aerogels. A preferred flow agent or glidant is silicon dioxide.

In a preferred embodiment, the controlled-release tablet will be made using an ingredient which acts as both a binder and flow agent (or glidant). A suitable source of such an ingredient is Prosolv SMCC 90 sold by Penwest. Prosolv SMCC 90 contains microcrystalline cellulose and lactose bound to a small percentage of silicon dioxide.

A tablet having sufficient mechanical strength and an acceptable release profile can be produced by mixing the active agents in powedered form with HPMC and suitable binders, lubricants and flow agents and compressing the mixture in a tablet press. A typical compression force used in forming the tablets is in the range of about 45 to about 56 KN, preferably about 50 to about 53 KN, to achieve a tablet having a hardness in the range of about 15 kp to about 30 kp, preferably about 18 kp to about 25 kp.

Time-Specific Formulations

As discussed above, in one embodiment, the anticholinergic agent(s) and the first and second antihistamines can be delivered using a single dosage formulation, in a time-specific manner. The time-specific controlled release dosage formulations can be any formulations which release one of the antihistamines at one time, and the other of the antihistamines at a later time. Such time-specific formulations are well-known to those of skill in the art, though not for administration of the specific formulations described herein.

In one embodiment, the formulations include (1) a core including a non-sedative formulation (or relatively less-sedative formulation), and (2) a swellable polymeric coating layer substantially surrounding the core including a sedative formulation. The swellable polymeric coating layer delays the release of the non-sedative formulation from the core for a predetermined period of time dependent upon the thickness of the swellable polymeric coating layer, to effect delivery of the pharmaceutically active agents in the non-sedative formulation at about the time of awakening.

The core of the time-specific formulation of the present invention may also include one or more pharmaceutically acceptable excipients in addition to the pharmaceutically active agent. Pharmaceutically acceptable excipients which may be employed are well known to those skilled in the art and include any conventional pharmaceutically acceptable tabletting excipients. Examples of suitable excipients include but are not limited to microcrystalline cellulose, dibasic calcium phosphate dihydrate, starch, sodium starch glycolate, crospovidone, croscarmellose sodium, magnesium stearate, lactose, maleic acid, colloidal silicon dioxide, talc, and glyceryl behenate.

In one particularly preferred embodiment, the core of the time-specific formulation includes, in addition to the pharmaceutically acceptable agents, a disintegration enhancing agent. The disintegration enhancing agent accelerates the disintegration of the core once the swellable polymeric coating layer is removed by dissolution or erosion. The disintegration enhancing agent provides the advantage that the pharmaceutically active agent is more readily delivered to the system by virtue of the faster disintegration of the core. The faster delivery of the pharmaceutically active agent to the system which results from the presence of the disintegration enhancing agent within the core advantageously produces a “spike” in the level of pharmaceutically active agent in the system. Thus, in the embodiment wherein the disintegration enhancing agent is present in the core, the pharmaceutically active agent is delivered substantially faster which causes the level of pharmaceutically active agent in the system to rapidly reach the maximum level, rather than more slowly as a stream which gradually reaches the maximum level of delivered drug. Suitable disintegration enhancing agents for use in the methods of the present invention include pharmaceutically acceptable excipients capable of generating effervescence. Specific examples of suitable disintegration enhancing agents include but are not limited to food acids, such as citric acid, tartaric acid, fumaric acid, maleic acid, succinic acid, and the like; acid anhydrides, such as succinic anhydride, fumaric anhydride, and the like; acid salts such as sodium dihydrogen phosphate, disodium dihydrogen pyrophosphate, sodium dihydrogen citrate, disodium hydrogen citrate, and the like; and carbonates such as sodium bicarbonate, sodium carbonate, potassium bicarbonate, potassium carbonate, sodium sesquicarbonate, calcium carbonate, glycine sodium carbonate, L-lysine carbonate, arginine carbonate, and the like.

The core can be prepared by any suitable tabletting technique known to those skilled in the art. For example, the pharmaceutically active ingredient may be admixed with the excipient(s) and, if present, the disintegration enhancing agent, and advantageously formed into a tablet using a conventional tabletting press, or dosed into a capsule.

The preformed core is substantially surrounded by a swellable polymeric coating layer. The swellable polymeric coating layer typically includes a hydrophilic gelling polymer or “swellable polymer” which swells on contact with gastro-intestinal juices to form a continuous film surrounding the core. The coating layer must sufficiently protect the integrity of the core for the desired period of time, without regard to the pH of the medium to which it is subjected. Once the desired, pre-delivery time period has elapsed, the core may, in one embodiment, be capable of relatively quick disintegration so that the pharmaceutically active agent is released as quickly as possible at the predetermined delivery time. Thus, it is desirable that the formulation be capable of the fast, time-specific release of the pharmaceutically active agent. The polymeric coating layer may be comprised of any suitable hydrophilic gelling polymer known to those skilled in the art. For example, suitable hydrophilic gelling polymers include but are not limited to cellulosic polymers, such as methylcellulose, carboxymethylcellulose, hydroxypropylcellulose, hydroxypropylmethylcellulose, hydroxyethylcellulose, and the like; vinyl polymers, such as polyvinylpyrrolidone, polyvinyl alcohol, and the like; acrylic polymers and copolymers, such as acrylic acid polymer, methacrylic acid copolymers, ethyl acrylate-methyl methacrylate copolymers, and the like; and mixtures thereof. Currently, the preferred swellable polymeric coating layer comprises hydroxypropylmethylcellulose.

Alternatively, the swellable polymeric coating layer may be comprised of other substances which are capable of becoming freely permeable with exactly defined kinetics following hydration in aqueous fluids. Such substances include polysaccharides, such as gelatin, saccharose, sorbitol, mannaese, and jaluronic acid; polyaminoacids; polyalcohols; polyglycols; and the like

In addition to the foregoing, the swellable polymeric coating layer may also include additional excipients such as lubricants, flow promoting agents, plasticizers, antisticking agents, natural and synthetic flavorings and natural and synthetic colorants. Specific examples of additional excipients include polyethylene glycol, polyvinylpyrrolidone, talc, magnesium stearate, glyceryl behenate, stearic acid, and titanium dioxide.

The swellable polymeric coating layer may be applied to the core using conventional film (or spray) coating techniques, double press coating or by the inventors' new method involving the alternate application of binder and powdered polymeric coating particles. In one preferred embodiment, the swellable polymeric coating layer is applied using film coating techniques whereby the hydrophilic gelling polymer is solubilized in an aqueous solution.

Although some organic solvents may be employed in the film coating application of the swellable polymeric coating layer, the inclusion of organic solvents in the film coating solution utilized in the methods of the present invention is not required.

The solution of hydrophilic gelling polymer can be applied to the core by any means of film coating including but not limited to fluid bed, or pan coating. Preferably, the solution of polymer is sprayed on the core to form the swellable polymeric coating layer.

The polymer is applied on the core (preferably by film-coating) in order to build the desired thickness of the swellable polymeric coating layer. For example, in the embodiment wherein film coating is employed, the core is sprayed with the solution of polymer until the desired thickness of swellable polymeric coating layer is achieved.

In another preferred embodiment, the swellable polymeric coating layer is applied to the core by an alternating two-step application of a binder solution and powdered polymeric coating particles. In the first step, the core is wet with the binder solution which serves to adhere the powdered polymeric coating particles to the core. Suitable binder solutions may include conventional pharmaceutically acceptable binder agents solubilized in a suitable solvent. Specific examples of binder agents include but are not limited to vinyl polymers, such as polyvinylpyrrolidone, polyvinyl alcohol, and the like; cellulosic polymers, such as hydroxypropylmethylcellulose, hydroxyethylcellulose, hydroxypropylcellulose, and the like; acrylic polymers and copolymers such as methacrylic acid copolymers, ethyl acrylate-methylmethacrylate copolymers, and the like; natural or synthetic gums, such as guar gum, arabic gum, xanthan gum, and the like; proteins or carbohydrates, such as gelatin, pectin, and the like; and mixtures thereof. Currently, polyvinylpyrrolidone is the preferred binder agent.

Suitable solvents for solubilizing the binder agents include solvents which are capable of substantially completely solubilizing the specific binder agent(s) selected and which are pharmaceutically and biologically acceptable for ingestion. Suitable solvents will be readily determinable by those skilled in the art. Water is currently the preferred solvent for solubilizing the binder agent, as it is pharmacologically and biologically well suited for ingestion. However, other examples of suitable solvents will be appreciated by those skilled in the art and are contemplated by the methods of the present invention.

The binder solution should be of sufficient viscosity to enable the wetting of the cores by any suitable wetting technique known to those skilled in the art. For example, the cores may be wetted with the binder solution by rotating the cores in a bath containing the binder solution. The cores may be suitably wetted by manual application of the binder solution by ladling the binder solution over the cores as the cores are rotating in a conventional coating pan. Alternatively, the cores may be wetted by spraying the binder solution on the cores. In one embodiment, the wetting step is advantageously carried out using conventional automated pan coating equipment wherein the cores are sprayed with the binder solution while rotating in the pan.

To provide the coating layer, the wetted cores are coated with dry, powdered polymeric coating particles which adhere to the binder-wetted core due to the presence of the binder on the surface of the core. The polymeric coating particles typically comprise a hydrophilic gelling polymer or “swellable” polymer which swells on contact with gastro-intestinal juices to form a continuous film surrounding the core, as described herein above. Currently, the preferred powdered polymeric particles comprise hydroxypropylmethylcellulose.

Hydroxypropylmethylcellulose is a polymer which is available in many forms, including forms of different molecular weight, extremely different viscosity and different substitution grade. Mixtures or blends of two or more different forms of hydroxypropylmethylcellulose can be used. Representative types of hydroxypropylmethylcellulose include METHOCEL E5® and METHOCEL K15M®, both of which are commercially available from Colorcon. The term “hydroxypropylmethylcellulose” as used herein, including the claims, refers to either a single hydroxypropylmethylcellulose or a blend of two or more forms of the polymer.

The polymer(s) of the swellable polymeric coating layer partially hydrates on the outer surface thereof after ingestion to form a gel-like layer that acts as a skin, controlling the rate of erosion of the coating layer. As a consequence, the release or delivery of the pharmaceutically active agent contained within the core is inhibited for the predetermined period of time.

After the powdered polymeric coating particles are applied to the wetted core, the steps of first, wetting the core with binder and second, coating with the powdered polymeric coating particles are repeated sequentially one or more additional times in order to build the desired thickness of the swellable polymeric coating layer around the core. In other words, the alternating steps of wetting the core and coating with the powdered polymeric coating particles are repeated in alternate fashion so that prior to each application of the powdered coating particles, the core is first wetted with the binder solution. In this manner, the repeated applications of binder solution and powdered polymeric coating particles build or increase the thickness of the swellable polymeric coating layer to the desired measure. The number of repeated wetting and coating cycles is dependent upon the desired delivery time of the pharmaceutically active agent. The thicker the swellable polymeric coating layer around the core, the longer the latency or lag time prior to delivery of the pharmaceutically active agent.

The swellable layer may also be applied by double-press coating, also known as compression-coating. The main advantage in comparison with the film-coating or the sugar-coating procedure is the elimination of water or other solvents during manufacturing. The manufacturing scheme normally starts with the loading of the bottom layer into the die from the hopper, then the core is centered on the bed of coating, this operation is followed by the deposition of the top layer of the coating. Finally, the whole is compressed by passing the punches between the compression rolls.

Irrespective of the method of application, the swellable polymeric coating layer is typically applied to the core to achieve the desired predetermined thickness of swellable polymeric coating. The desired predetermined thickness of the swellable polymeric coating layer is dependent upon the desired lag time or delay prior to delivery of the pharmaceutically active agent. The thicker the swellable polymeric coating layer around the core, the longer the latency, or lag time prior to delivery of the agent. Typically, the swellable polymeric coating layer is applied to a thickness sufficient to achieve a weight gain of between about 5 and about 500 percent, preferably between about 10 and about 200 percent as determined by solid substance. The swellable polymeric coating layer is sufficiently thick to provide a core:coating layer weight ratio of between about 20:1 and about 1:5 inclusive, or a thickness in excess of about 10 μm up to about 3 mm, inclusive. Preferably, the swellable polymeric coating layer is sufficiently thick to achieve a core:coating layer weight ratio of between about 5:1 and about 1:3 inclusive, or a thickness of between about 50 μm and about 1500 μm.

### III. Methods of Treating Rhinitis

The compositions can be used to treat allergic rhinitis, as well as to treat other indications for which antihistamines are used. Suitable patient populations for which the methods and formulations of the present invention are directed include mammals in general, and in particular, humans.

The regimens include daytime and nighttime formulations, each of which comprises an anticholinergic and an antihistamine. The daytime formulation and nighttime formulation either include different antihistamines, or include different dosages of the same antihistamine.

Where different dosages of the same antihistamine are used, the nighttime formulation can include a relatively higher dosage of an antihistamine than used in the daytime formulation. For example, a sedating antihistamine can be used in the nighttime formulation and a lower dose of the antihistamine can be used during the day. In this manner, one can largely avoid sedation during the day. To the extent the dosage of the antihistamine is lower during the day, a decongestant can be added to the daytime formulation, so long as there is a balance between the sedative properties of the antihistamine and the stimulatory properties of the decongestant, ideally such that the formulation is neither overly stimulatory nor overly sedative.

Where different antihistamines are used in the daytime and nighttime formulations, in one aspect of this embodiment, a second generation antihistamine is used in the daytime formulation, and a first generation antihistamine is used in the nighttime formulation. In this manner, one can avoid sedation during the day, but provide sedation at night. There may also be a different dosage between the first and second generation antihistamines, depending on the effective dosage for the selected antihistamines. In another aspect of this embodiment, the first antihistamine is less active than the second antihistamine. The term “less active” is used herein to describe lower dosages of the same antihistamine, with respect to the first embodiment, or with respect to the second embodiment, an antihistamine with less antagonistic activity at the histamine H1 receptor.

In one aspect of this embodiment, the second generation antihistamine is not acrivastine, astemizole, cetirizine, loratadine, mizolastine or fexofenadine. Because the effective dosage of different antihistamines differs depending on the antihistamine, the amount of antihistamine in each of the daytime and nighttime formulations may be the same or different.

In both of these embodiments, the therapeutic regimens include indicia for distinguishing between the first and second pharmaceutical dosage forms, where the indicia distinguishes the first pharmaceutical dosage form for daytime administration and the second pharmaceutical dosage form for nighttime administration. A prepackaged dispenser pre-filled with the pharmaceutical dosage forms and including the indicia is also provided.

In a third embodiment, rather than using a combination of a daytime and a nighttime formulation, the methods involve administering a composition in a single dosage form, which composition provides for sustained release of an anticholinergic agent, release of a first antihistamine for an initial twelve hour period, and delayed release of a second antihistamine for a second twelve hour period. This can be accomplished, for example, using a drug delivery device that includes a core and a coating, wherein the first antihistamine is present in the coating, and the second antihistamine is present in the core, with the anticholinergic agent present in both the core and the coating. The terms “first antihistamine” and “second antihistamine,” in this context, refer to either the differing dosages of the same antihistamine, or to a first generation and a second generation antihistamine, as discussed above with respect to the daytime and nighttime formulations. Thus, in this embodiment, one can achieve the same benefits as provided by taking both the daytime and nighttime formulations, but need only to take a single dose. The single dose form may be formulated for administration at night, by including a sedating antihistamine in the coating for instantaneous release and a non-sedating antihistamine in the core for delayed release. The single dose form may also be formulated for administration during the day, with a non-sedating antihistamine in the coating for initial sustained release, and a sedating antihistamine in the core for delayed sustained release. Both coating and core contain an anticholinergic for continuous sustained release. This embodiment includes a prepackaged dispenser with indicia describing the time of day for administration, depending on which antihistamine is initially released.

These therapeutic regimens can be used to treat the symptoms of a rhinitic condition. The presence of the anticholinergic in both the daytime and nighttime aids in suppressing post-nasal drip throughout the day and night. The evening administration of the anticholinergic agent can help avoid the potential complications of having mucous, particularly mucous infected with bacteria and/or viruses, enter the bronchial tubes and result in bronchitis. The daytime administration of the anticholinergic agent can help minimize post-nasal drip. Further, with respect to those embodiments where the daytime formulations include a second generation antihistamine, one difference between first and second generation antihistamines is that the first generation antihistamines tend to have some anticholinergic properties, but second generation antihistamines tend not to have such properties. Thus, the presence of an anticholinergic can provide anticholinergic properties that are lacking in the second generation antihistamine used in the daytime formulation.

Time-Specific Administration

In one embodiment, the compositions and methods described herein can be used to treat allergies occurring early in the morning by using a time-specific formulation. That is, in one embodiment, the formulation is prepared such that it is administered orally in one formulation (i.e., a single tablet), in such a manner that when the patient is asleep, a first composition is administered, and around the time the patient awakens, a second composition is administered. The composition administered when the patient awakens and/or is awake includes an anticholinergic agent and either a non-sedative antihistamine, or a lower dose of a sedative antihistamine. The composition orally administered before patient falls asleep, and which releases its active agents while the patient is asleep, includes a sedative antihistamine and an anticholinergic agent. Relative to the composition administered when the patient is awake, the antihistamine is either the same, but administered at a higher dosage, or is different. When the antihistamine is different, it either has more of a tendency to cause drowsiness, or has more activity at the histamine receptor. In this embodiment, a decongestant can optionally be present in the composition released during the daytime.

In this embodiment, the composition delivers a sedative antihistamine during the nighttime when the patient is asleep, and delivers a non-sedative antihistamine, or a lower dosage of a sedative antihistamine, when the patient is awake. In contrast to where the patient would wake up and need to take an antihistamine, the antihistamine is already administered, and working to prevent the patient from developing an adverse response to allergens, such as pollen, which are at relatively high levels early in the morning. This advantage can be achieved by administering the pharmaceutical formulation before the patient goes to sleep. The pharmaceutical formulations can delay the release of the pharmaceutically active agent so that the agent is delivered at about the last few hours of night or sleep or at the time of awakening to provide early morning relief of allergy symptoms. Although the formulation has been ingested prior to sleeping, the pharmaceutically active agents intended to treat the patient when he or she is awake are not delivered until about the last few hours or night-time or sleeping or the time of awakening so that the subject is not exposed to these pharmaceutically active agents throughout the entire night.

In one embodiment, the time-specific formulation for use in the methods described herein includes multiple time-specific dosage units. In one aspect of this embodiment, the first time-specific dosage unit includes (1) a core comprising the pharmaceutically active agent and a disintegration enhancing agent, and (2) a swellable polymeric coating layer substantially surrounding the core. The second time-specific dosage unit includes (1) a core comprising the pharmaceutically active agent without the disintegration enhancing agent, and (2) a swellable polymeric coating layer substantially surrounding the core. This multi-unit formulation is advantageous in that the presence of the disintegration enhancing agent in the first dosage unit causes the core of that unit of the formulation to more quickly disintegrate and release the pharmaceutically active agent more rapidly, compared to the second unit which does not include the disintegration enhancing unit. The core of the second unit disintegrates and releases the pharmaceutically active agent more slowly and gradually, as compared to the first unit. The disintegration of the first unit provides a quick delivery of the pharmaceutically active agent while the slower disintegration of the second unit provides a continuing delivery stream of pharmaceutically active agent. The result is a single pharmaceutical formulation which provides quick onset of the therapeutic benefits of the pharmaceutically active agent with prolonged effects of those benefits.

Examples of these types of devices are disclosed, for example, in U.S. Pat. No. 5,788,987, and U.S. Pat. No. 5,958,458, the contents of which are hereby incorporated by reference in their entirety.

### III. Methods of Preparing Formulations

In addition to the therapeutic regimens, and methods of treatment using the regimens, methods for formulating rhinitic treatment regimens are also provided. In one embodiment, the formulation method comprises producing a first pharmaceutical dosage form that includes a therapeutically effective amount of a first antihistamine and a therapeutically-effective amount of an anticholinergic agent, and a second pharmaceutical dosage form comprising a therapeutically-effective amount of a second antihistamine, and a therapeutically effective amount of the anticholinergic. The formulations are intended for use in a manner in which the first pharmaceutical dosage form is intended to be administered during the day, and the second pharmaceutical dosage form is intended to be administered during the evening. So that the proper formulations are administered, the formulations are marked with indicia for daytime and nighttime administration.

As discussed above, the daytime and nighttime formulations either include different antihistamines, or include different dosages of the same antihistamine. Where different dosages of the same antihistamine are used, the nighttime formulation can include a relatively higher dosage of an antihistamine, for example, a sedating antihistamine, than the daytime formulation. To the extent the dosage of the antihistamine is lower during the day, a decongestant can be added to the daytime formulation. Where different antihistamines are used in the daytime and nighttime formulations, in one aspect of this embodiment, a second generation antihistamine is used in the daytime formulation, and a first generation antihistamine is used in the nighttime formulation. In another aspect of this embodiment, the first antihistamine is less active than the second antihistamine. The term “less active” is used herein to describe lower dosages of the same antihistamine, with respect to the first embodiment, or an antihistamine with less antagonistic activity at the histamine H1 receptor, in the second embodiment.

In one aspect of this embodiment, the second generation antihistamine is not acrivastine, astemizole, cetirizine, loratadine, mizolastine or fexofenadine. Because the effective dosage of different antihistamines differs depending on the antihistamine, the amount of antihistamine in each of the daytime and nighttime formulations may be the same or different.

The methods for formulating these regimens further include providing indicia for distinguishing between the first and second pharmaceutical dosage forms, where the indicia distinguishes the first pharmaceutical dosage form for daytime administration and the second pharmaceutical dosage form for nighttime administration. A prepackaged dispenser pre-filled with the pharmaceutical dosage forms and including the indicia is also provided.

Alternatively, one can formulate a single dosage form which provides for sustained release of an anticholinergic agent, release of a first antihistamine for an initial twelve hour period, and delayed release of a second antihistamine for a second twelve hour period. This can be accomplished, for example, by formulating a drug delivery device that includes a core and a coating, wherein the first antihistamine is present in the coating, and the second antihistamine is present in the core, with the anticholinergic agent present in both the core and the coating.

The present invention provides for a prefilled, pharmaceutical dosage form dispenser containing at least two compositionally different pharmaceutical dosage forms for the treatment of rhinitis and indicia for distinguishing the pharmaceutical dosage forms and signifying their use together. Either single or multiple dispensers of each pharmaceutical dosage form may be contained as is desired. The pharmaceutical dosage forms may be in the form of tablets, capsules, lozenges or gelcaps, some of which may require reconstituting, or any generally recognized oral form of medication.

Subjects suffering from rhinitic conditions generally display pale or violaceous nasal mucosa because of the engorged veins, nasal polyps, swelling of the eyelids, injected sclerae (the whites of the eyes may be red), allergic shiners (darkened areas under the lower eyelids thought to result from venous pooling of blood), and extra skin folds in the lower eyelids.

Factors which may cause rhinitis include year-round allergens, for example, dust mites, cockroaches, molds and animal dander, and seasonal outdoor allergens, for example, tree, grass and ragweed pollens. Indoor allergens include, for example, mold spores and animal allergens.

The formulation of therapeutically effective combinations requires pharmaceutical expertise and understanding of the pharmacokinetic and pharmacodynamic actions and side effects of antihistamines, anticholinergics, and other formulated components, including components which may affect the pharmacokinetic and pharmacodynamic bioavailability and effectiveness of the active ingredients, and their suitability of use together. It is therefore another embodiment to provide a subject with an effective regimen that maximizes the pharmacokinetic and pharmacodynamic bioavailability and effectiveness of the regimen.

It is another embodiment to provide a method and device for organizing, storing, and coordinating regimens for the treatment of rhinitis for the purpose of convenience in using such regimens by providing such regimens, prepackaged in such a way that incorporates coordinating indicia and administration of the pharmaceutical dosage forms.

It is another embodiment to provide regimens which provide a therapeutic combination of anticholinergic and an antihistamine dosage during the night in accordance with its pharmacokinetic and pharmacodynamic profile so as to achieve histamine receptor binding in the morning hours, at the time of awakening.

In one embodiment, a user is provided with a therapeutic combination of anticholinergic and attenuated first antihistamine H₁ receptor activity during the day, which is suitable with respect to its half-life, duration of action, and duration of side effect, and a dosing of a second antihistamine having greater H₁ receptor activity than the first antihistamine with the anticholinergic at nighttime.

In one embodiment, a pre-filled, pharmaceutical dosage form dispenser is provided containing at least two compositionally different pharmaceutical dosage forms for the treatment of rhinitis, and indicia for distinguishing the pharmaceutical dosage forms. The dispenser can have one of any number of forms, including, but not limited to, a box with the dosage forms in bottles, a blister package, a box of individual blister packages, or a box of pouches.

The embodiments of the dispenser will become apparent in light of the following drawings and detailed description thereof.

Referring to the drawings, it will be understood that while various embodiments of the invention have been illustrated and described, the invention is not limited to such embodiments. Changes and additions may be made therein and thereto without departing from the spirit of the invention.

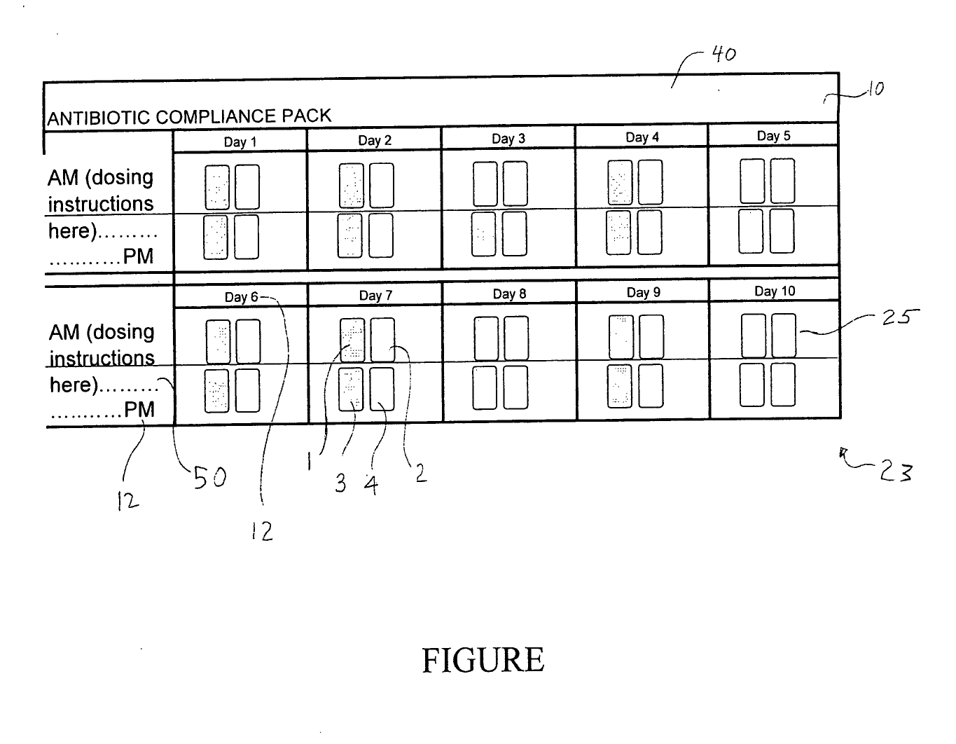

Referring to FIG. 1 of the drawings, the container is in the form of a blister pack dispenser comprising a frontside surface 10 with indicia 12 defined thereon; a blister film 20 affixed to the frontside surface 10 having held therein two types of discrete pharmaceutical dosage forms 1 and 3.

The blister pack dispenser shown in FIG. 1 is an elongate rectangle. The blister pack dispenser may be of any acceptable shape. The indicia 12 is defined on the frontside surface 10 by the arrangement of printed words or other symbols along two axes, the first axis being alongside one edge of the frontside surface 10, the other axis being at right angles to the first. The first axis is defined by the words “Day 1”, “Day 2”, etc., and may be regularly spaced, these words representing, for example, consecutive days or administration periods of the therapy or regimen. The other axis may be defined by the words “AM” and “PM”, or other well recognized equivalent symbols representing daytime and night-time within each day/period of the therapy or regimen.

The front side surface 10 has an area 50, outside the indicia 12, on which may be printed administration instructions. Administration instructions may include words or well recognized symbols that convey to the user the intended administration therapy or regimen. Alternatively, the section may be intentionally left blank, for example, to allow a health care provider to write specific instructions.

The blister film generally indicated at 20 may be of a conventional blister pack dispenser type, in which blisters 25 may be formed in a regular grid of rows and columns.

The grid of blisters 25 may be so arranged in the blister film 20 that a column of blisters 25 is in register with each of “Day 1”, “Day 2”, etc. on the front side surface 10 and rows of blisters are in register with “AM” and with “PM” on the front side surface 10.

The two types of discrete pharmaceutical dosage forms generally indicated at 1 and 3 may be located in the closed blisters 25 of the blister film 20. Pharmaceutical dosage forms 1 may comprise a composition comprising an anticholinergic and an attenuated dosage of the first antihistamine and packed in the two rows of blisters 25 in register with the word “AM” in the indicia 12. Pharmaceutical dosage forms of the pharmaceutical dosage form 3 that include the second antihistamine, which may be sedative, and the anticholinergic are packed in the rank of blisters 25 in register with the word “PM” in the indicia 12. The pharmaceutical dosage forms of the pharmaceutical dosage form 1 may be of a different color to those of pharmaceutical dosage form 3.

The order of packing of the pharmaceutical dosage forms of types 1 and 3 located in the blisters 25 of the blister film 20 in register with the indicia 12, and the different colors of the two dosage types facilitate the taking of pharmaceutical dosage forms 1 by day and pharmaceutical dosage form 3 at night.

In addition to indicating and facilitating the taking of the various pharmaceutical dosage forms in accordance with a desired treatment regime, the dispenser illustrated also conveniently shows when the necessary pharmaceutical dosage forms have been taken.

To remove any pharmaceutical dosage form 1 and 3 at a time indicated as appropriate as above the corresponding blister 25 containing it may be accomplished by pressing with a finger to push the pharmaceutical dosage form through the front side surface 10.

The base and blister film of the blister pack dispenser may be of any materials suitable for the construction of blister packs, for example, an aluminum foil base and a thermoplastic blister film.

Although the administration instructions may be printed on the base, they may also be written or printed on a separate surface such as a sheet of paper, or on a label attached to the pack.

Although the illustrated dispenser specifically described is generally for up to a five-day dosage regime, it is envisaged that the dispenser may be adapted for longer or shorter periods of time, as desired, merely by shortening or lengthening the dispenser and correspondingly decreasing or increasing the number of columns of blisters as appropriate.

Further, although the illustrated dispenser specifically described is for a regime of a pharmaceutical dosage forms for day-time use and for night-time use, it is envisaged that the desired regime may specify any number of pharmaceutical dosage forms for each aspect of the therapy. Consequently, the dispenser may be adapted in accordance with the requirements of the regime by narrowing or widening the dispenser and correspondingly decreasing or increasing the number of rows of blisters and the number of rows in register with “AM” and “PM” as appropriate.

The blister pack dispenser described has an indicia defined on it in the form of rows and columns with corresponding positioning of the pharmaceutical dosage form containing blisters. The indicia, and corresponding blisters, may be in any geometric configuration provided that the indicia clearly indicates which pharmaceutical dosage forms are to be taken during the day and which pharmaceutical dosage forms are to be taken at night. Also, the indicia may be omitted, but in this case pharmaceutical dosage forms of the different types must have a visible distinguishing feature, such as a difference in color, to indicate that they relate to different aspects of the dosage regime. The indicia and such a distinguishing feature may both be present.

One or more blister packs may be housed in any suitable form for dispensing.

The dispenser embodiments described herein are not limited to blister packs. Thus, any conventional pharmaceutical containers are suitable. Examples thereof include bottles, tubes, canisters and packets. Where such containers do not readily permit the housing of the pharmaceutical dosage forms in register with a indicia, for example bottles, the pharmaceutical dosage forms must be mutually distinguished by some visible feature, such as a difference in color, form, shape or size, or by marks or printing therein, to indicate which pharmaceutical dosage forms are for day-time and which pharmaceutical dosage forms are for night-time.

The packaging may contain combinations of medications, which include anticholinergics and antihistamines, which comprise a regimen for treating rhinitis. For example, the packaged medication may be comprised of at least two pharmaceutical dosage forms, one for administration when sedation is not desired, as, for example, during the day, and one for administration in anticipation of peak histamine release and/or when sedation is desired, as, for example, at night. The regimen is devised utilizing a combination of pharmaceutical dosage forms which are favorable for use with each other, particularly with regard to pharmacokinetic and therapeutic characteristics. It is possible to formulate these regimens with presently available pharmaceutical dosage forms or active agents as well as with newly formulated pharmaceutical dosage forms or active agents. Examples of regimens, some of which employ presently available pharmaceutical dosage forms and active agents, are described below.

There are a number of possible combinations of anticholinergics and antihistamines presently available that can be employed in the present invention. Other medications, such as analgesics and non-sympathomimics may be incorporated into the dosage forms.

The embodiments of the treatment regimen will become apparent in light of the following examples. The following examples are illustrative and not to be interpreted as limiting or restrictive. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements.

### EXAMPLE 1

A first pharmaceutical dosage form comprising about 2.5 milligrams methscopolamine nitrate and about 4 milligrams of chlorpheniramine maleate may be provided in tablet form to be taken in the morning. This tablet may be colored white, for example. A second pharmaceutical dosage form of about 2.5 milligrams methscopolamine nitrate and about 8 milligrams of chlorpheniramine maleate may be provided in tablet form to be taken at bedtime. This tablet may be colored blue. The amount of chlorpheniramine maleate in the second tablet therefore would provide greater H₁ receptor activity.

### EXAMPLE 2

A first pharmaceutical dosage form comprising about 2.5 milligrams methscopolamine nitrate (anticholinergic) and about 6 milligrams brompheniramine maleate (antihistamine) may be provided in tablet form to be taken in the morning. This tablet may be colored white, for example. A second pharmaceutical dosage form of about 2.5 milligrams methscopolamine nitrate and about 25 milligrams diphenhydramine hydrochloride may be provided in tablet form to be taken at bedtime. This tablet may be colored blue, for example. The amount of diphenhydramine hydrochloride in the second tablet would provide for greater H₁ receptor activity than the amount of brompheniramine maleate in the first tablet.

### EXAMPLE 3

A first pharmaceutical dosage form comprising about 2.5 milligrams methscopolamine nitrate and about 3 milligrams dexchlorpheniramine maleate (antihistamine) may be provided in tablet form to be taken in the morning. This tablet may be colored white, for example. A second pharmaceutical dosage form of about 2.5 milligrams methscopolamine nitrate and about 6 milligrams dexchlorpheniramine maleate may be provided in tablet form to be taken at bedtime. This tablet may be colored blue, for example. The amount of dexchlorpheniramine maleate in the second tablet therefore would provide greater H₁ receptor activity.

### EXAMPLE 4

A first pharmaceutical dosage form comprising about 1.25 milligrams methscopolamine nitrate and about 2 milligrams chlorpheniramine maleate (antihistamine) may be provided in tablet form to be taken in the morning. This tablet may be colored white, for example. A second pharmaceutical dosage form of about 1.25 milligrams methscopolamine nitrate and about 4 milligrams chlorpheniramine maleate hydrochloride may be provided in tablet form to be taken at bedtime. This tablet may be colored blue, for example. The amount of chlorpheniramine maleate in the second tablet therefore would provide greater H₁ receptor activity.

### EXAMPLE 5

A first pharmaceutical dosage form comprising about 2.5 milligrams methscopolamine nitrate and about 5 milligrams desloratidine (antihistamine) may be provided in tablet form to be taken in the morning. This tablet may be colored white, for example. A second pharmaceutical dosage form of about 2.5 milligrams methscopolamine nitrate and about 8 milligrams chlorpheniramine maleate may be provided in tablet form to be taken at bedtime. This tablet may be colored blue, for example. The antihistamine in the second pharmaceutical dosage form would have a greater sedative effect than the first pharmaceutical dosage form.

### EXAMPLE 6

A first pharmaceutical dosage form comprising about 2.5 milligrams methscopolamine nitrate and about 180 milligrams fexofenadine (antihistamine) may be provided in tablet form to be taken in the morning. This tablet may be colored white, for example. A second pharmaceutical dosage form of about 2.5 milligrams methscopolamine nitrate and about 25 milligrams diphenhydramine hydrochloride may be provided in tablet form to be taken at bedtime. This tablet may be colored blue, for example. The antihistamine in the second pharmaceutical dosage form would have a greater sedative effect than the first pharmaceutical dosage form. Despite an indication for 4 to 6 hour dosing for chlorpheniramine, a single dose may inhibit the symptoms of rhinitis for more than 24 hours. Maximal pharmacological sedation peaks approximately 3 to 4 hour after dosing and sedation persists usually not longer than 6 to 8 hours following dosing.

The combination of an attenuated antihistamine dosing and anticholinergic during the day and greater pharmacologic antihistamine dosing with anticholinergic dosing at night pharmacologically maximizes the short duration of sedation in relation to the longer duration of rhinitic symptom suppression while avoiding the risk of hypertension (EXAMPLES 1-4). This combined dosage regimen anticipates peak morning histamine release and effectively confers combined antihistamine and anticholinergic activity during the day with minimal sedation as a result of pharmacologically adjusted dosing of the first antihistamine during the day.

Alternatively, the combination of non-sedative antihistamine dosing and anticholinergic during the day and sedative antihistamine dosing with anticholinergic dosing at night reduces or eliminates daytime sedation in relation to the longer duration of rhinitic symptom suppression while avoiding the risk of hypertension (EXAMPLES 5-6). The absence of a sympathomimetic decongestant dosing avoids the potential for hypertension stimulation and insomnia at night. This combined dosage regimen anticipates peak morning histamine release and effectively confers combined antihistamine and anticholinergic activity during the day with minimal sedation as a result of pharmacologically reducing or eliminating sedation with the first antihistamine dosage during the day while pharmacologically providing a longer duration of rhinitic symptom suppression with the second antihistamine dosage.

Other variations may occur to those skilled in the art which are within the scope of the invention as set forth in the appended claims. Those of skill in the art may also recognize modifications to these presently disclosed embodiments. These variations and modifications are meant to be covered by the spirit and scope of the present claims.

We claim:

1. A therapeutic regimen for treating rhinitis, comprising:
a) a daytime formulation comprising an anticholinergic agent and a first antihistamine, and
b) a nighttime formulation comprising an anticholinergic agent and a second antihistamine,
wherein the first antihistamine and the second antihistamine are either different antihistamines, or different dosages of the same antihistamine.

2. The therapeutic regimen of claim 1, wherein the daytime formulation comprises a second or third generation antihistamine, and the nighttime formulation comprises a first generation antihistamine.

3. The therapeutic regimen of claim 1, wherein the daytime formulation and the nighttime formulation both comprise a first generation antihistamine, wherein the dosage of the antihistamine in the daytime formulation is attenuated to lower the amount of sedation relative to the nighttime formulation.

4. The therapeutic regimen of claim 1, wherein the daytime formulation and the nighttime formulation both comprise a first generation antihistamine, wherein the daytime formulation further comprises a decongestant which lowers the amount of sedation relative to the nighttime formulation.

5. The therapeutic regimen of claim 4, wherein the amount of the decongestant is sufficient to lower the amount of sedation relative to the nighttime formulation, and is provided in an amount suitable to cause the overall formulation to not be stimulating for the majority of patients.

6. The therapeutic regimen of claim 1, wherein the formulation is substantially devoid of a decongestant.

7. The therapeutic regimen of claim 1, wherein the antihistamine in the nighttime formulation is chlorpheniramine maleate.

8. The therapeutic regimen of claim 1, wherein the anticholinergic agent is methscopolamine.

9. The therapeutic regimen of claim 1, wherein the antihistamine in the daytime formulation is not acrivastine, astemizole, cetirizine, loratadine, mizolastine or fexofenadine.

10. The therapeutic regimen of claim 1, wherein the first antihistamine has less antagonistic activity at the histamine H1 receptor than the second antihistamine.

11. The therapeutic regimen of claim 1, further comprising indicia for distinguishing between the daytime and nighttime formulations.

12. The therapeutic regimen of claim 1, further comprising packaging that separates the daytime from the nighttime formulations.

13. The therapeutic regimen of claim 12, wherein the packaging comprises a blister package.

14. A method of treating rhinitis, comprising administering a formulation of any of claims 1-13 to a patient in need of treatment thereof.

15. A method of formulating a therapeutic regimen for treating rhinitis, comprising:
a) producing a first pharmaceutical dosage form that includes a therapeutically effective amount of a first antihistamine and a therapeutically-effective amount of an anticholinergic agent, and a second pharmaceutical dosage form comprising a therapeutically-effective amount of a second antihistamine, and a therapeutically effective amount of the anticholinergic agent,
wherein the first and second antihistamines are either different antihistamines, or different dosages of the same antihistamine, and wherein one of the first and second dosage forms is a daytime formulation, and the other is a nighttime formulation,
b) providing the dosage forms in packaging that with indicia that distinguishes the daytime formulation from the nighttime formulation, and
c) providing instructions for using the treatment regimen so as to administer the first pharmaceutical dosage form during the day, and the second pharmaceutical dosage form during the evening.

16. The method of claim 15, wherein the daytime and nighttime formulations comprise different dosages of the same antihistamine.

17. The method of claim 15, wherein the daytime and nighttime formulations comprise different antihistamines.

18. The method of claim 15, wherein the daytime formulation comprises a second or third generation antihistamine, and the nighttime formulation comprises a first generation antihistamine.

19. The method of claim 15, wherein the daytime formulation comprises an antihistamine which has less antagonistic activity at the histamine H1 receptor than the antihistamine in the nighttime formulation.

20. The method of claim 15, wherein the daytime formulation comprises a second generation antihistamine, wherein the second or third generation antihistamine is not acrivastine, astemizole, cetirizine, loratadine, mizolastine or fexofenadine.

21. A therapeutic regimen for treating rhinitis, comprising, in a single dosage form:
a) a daytime formulation comprising an anticholinergic agent and an antihistamine, and
b) a nighttime formulation comprising an anticholinergic agent and an antihistamine,
wherein the daytime formulation and nighttime formulation either include different antihistamines, or include different dosages of the same antihistamine, and
wherein the dosage form provides for sustained release, over an initial period, of the daytime formulation, and sustained release, delayed until after the initial period, of the nighttime formulation, or, alternatively, sustained release, over an initial period, of the nighttime formulation, and
sustained release, delayed until after the initial period, of the daytime formulation.

22. The therapeutic regimen of claim 21, wherein the dosage form comprises:
(1) a core including the following active agents:
a) a first antihistamine,
b) an anticholinergic agent, and
c) optionally, a decongestant, and
(2) a swellable polymeric coating layer substantially surrounding the core; comprising:
a) a second antihistamine and
b) an anticholinergic agent,
wherein the formulation is administered prior to sleep, and wherein the swellable polymeric coating layer delays the release of the pharmaceutically active agents from the core for a predetermined period of time dependent upon the thickness of the swellable polymeric coating layer, to permit delivery of the active agents at about the time of awakening and to treat the symptoms of allergic rhinitis that occur in the early morning,
and wherein the second dosage formulation comprising the second antihistamine is also administered in the evening, and delivers the majority of the second antihistamine during the evening while the patient is asleep.

22. The therapeutic regimen of claim 21, wherein the first antihistamine and the second antihistamine are different dosages of the same antihistamine.

23. The therapeutic regimen of claim 21, wherein the first and second antihistamines are different antihistamines.

24. The therapeutic regimen of claim 21, wherein the swellable polymeric coating layer comprises a hydrophilic swellable polymer selected from the group consisting of methylcellulose, carboxymethylcellulose, hydroxypropylcellulose, hydroxypropylmethylcellulose, hydroxyethylcellulose, polyvinylpyrrolidone, polyvinyl alcohol, acrylic acid polymer, methacrylic acid copolymers, ethyl acrylate-methyl methacrylate copolymers, natural rubbers, poloxamers, polysaccharides, and mixtures thereof.

25. The therapeutic regimen of claim 21, wherein the swellable polymeric coating layer is applied to the core by film coating.

26. The therapeutic regimen of claim 21, wherein the swellable polymeric coating layer is applied to the core by alternately (i) wetting the core with a binder solution and (ii) coating the core with powdered polymeric coating particles, a sufficient number of times to produce the desired thickness of swellable polymeric coating layer.

27. The therapeutic regimen of claim 26, wherein the binder solution is selected from the group consisting of polyvinylpyrrolidone, hydroxypropylmethylcellulose, polyvinyl alcohol, hydroxyethylcellulose, hydroxypropylcellulose, methylcellulose, methacrylic acid copolymers, ethyacrylate-methylmethacrylate copolymers, guar gum, arabic gum, xanthan gum, gelatine, pectin and mixtures thereof; and the powdered polymeric coating particles comprise a hydrophilic swellable polymer selected from the group consisting of methylcellulose, carboxymethylcellulose, hydroxypropylcellulose, hydroxypropylmethylcellulose, hydroxyethylcellulose, polyvinyolpyrrolidone, polyvinyl alcohol, acrylic acid polymer, methacrylic acid copolymers, ethyl acrylate-methyl methacrylate copolymers, natural rubbers, poloxamers, polysaccharides, and mixtures thereof.

28. The therapeutic regimen of claim 21, wherein the swellable polymeric coating layer comprises hydroxypropylmethylcellulose.

29. The therapeutic regimen of claim 21, wherein the swellable polymeric coating layer comprises a mixture of 1) hydroxypropylmethylcellose having a typical weight percent substitution corresponding to 29% methoxyl and 8% hydroxypropoxyl groups, and a nominal viscosity of 2% water solution at 20.degree. C. ranging from 3 to 100 mPa·s; and 2) hydroxypropylmethylcellulose having a typical weight percent substitution corresponding to 22.1% methoxyl and 8.1% hydroxypropoxyl groups, and a nominal viscosity of 2% water solution at 20.degree. C. ranging from 4,000 to 100,000 mPa·s.

30. The therapeutic regimen of claim 21, wherein the swellable polymeric coating layer is sufficiently thick to achieve a core:coating layer weight ratio of between about 20:1 and about 1:5.

31. The therapeutic regimen of claim 21, wherein the swellable polymeric coating layer is sufficiently thick to achieve a core:coating layer weight ratio of between about 5:1 and about 1:3.

32. The therapeutic regimen of claim 21, wherein the swellable polymeric coating layer is not less than about 50 μm thick.

33. The therapeutic regimen of claim 21, wherein the core further comprises a disintegration enhancing agent.

34. The therapeutic regimen of claim 33, wherein the disintegration enhancing agent is selected from the group consisting of citric acid, tartaric acid, fumaric acid, maleic acid, succinic acid, succinic anhydride, maleic anhydride, sodium dihydrogen phosphate, disodium dihydrogen pyrophosphate, sodium dihydrogen citrate, disodium hydrogen citrate, sodium bicarbonate, sodium carbonate, potassium bicarbonate, potassium carbonate, sodium sesquicarbonate, glycine sodium carbonate, calcium carbonate, L-lysine carbonate, and arginine carbonate.

35. The therapeutic regimen of claim 21, wherein the antihistamine to be administered during the daytime comprises a second generation antihistamine, and the antihistamine to be administered during the evening comprises a first generation antihistamine.

36. The therapeutic regimen of claim 21, wherein the antihistamine to be administered during the daytime has less antagonistic activity at the histamine H1 receptor than the antihistamine to be administered in the evening.

37. The therapeutic regimen of claim 21, wherein the daytime formulation comprises a second generation antihistamine, wherein the second generation antihistamine is not acrivastine, astemizole, cetirizine, loratadine, mizolastine or fexofenadine.

38. A method of treating rhinitis, comprising administering a formulation of any of claims 21-37 to a patient in need of treatment thereof.

39. The therapeutic regimen of claim 21, wherein indicia indicating the time of day for administration of the formulation is included.

40. A method of formulating a therapeutic regimen for treating rhinitis, comprising:
(a) producing in a single dose form,
a daytime formulation comprising an anticholinergic agent and an antihistamine, and, a nighttime formulation comprising an anticholinergic agent and an antihistamine,
wherein the daytime formulation and nighttime formulation either include different antihistamines, or include different dosages of the same antihistamine, and
wherein the dosage form provides for release, over an initial period, of the daytime formulation, and release, delayed until after the initial period, of the nighttime formulation, or, alternatively,
wherein the dosage form provides for release, over an initial period, of the nighttime formulation, and release, delayed until after the initial period, of the daytime formulation.

41. The method of claim 40, wherein the dosage form is provided in a package which comprises indicia indicating the time of day in which the formulation is to be administered.
